(19) United States
(12) Patent Application Publication
(10) Pub. No.: US 2008/0004689 A1
(43) Pub. Date: Jan. 3, 2008

(54) Systems and Methods for Making Medical Devices

(76) Inventors: Linda Jahnke, Burnsville, MN (US); Jeff Scheinost, Buffalo, MN (US); Daniel Breen, Blaine, MN (US)

Correspondence Address: FISH & RICHARDSON PC, P.O. BOX 1022, MINNEAPOLIS, MN, 55440-1022, US

(21) Appl. No.: 11/624,435

(22) Filed: Jan. 18, 2007

Related U.S. Application Data
(60) Provisional application No. 60/760,274, filed on Jan. 19, 2006.

(51) Int. Cl. A61F 2/82; A61F 2/06

(52) U.S. Cl. 623/1.15; 623/1.34

(57) ABSTRACT

Systems and methods for making medical devices are disclosed.

### CROSS-REFERENCE TO RELATED APPLICATIONS

This application claims the benefit of prior U.S. provisional application 60/760,274, filed Jan. 19, 2006.

### TECHNICAL FIELD

The invention relates to systems and methods for making medical devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprosthesis include stents and covered stents, sometimes called “stent-grafts”.

Endoprostheses can be delivered inside the body by a balloon catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon expandable endoprosthesis. During delivery, the balloon carrying the endoprosthesis is folded to a low profile, and subsequently, the balloon is inflated to deform and to fix the expanded endoprosthesis at a predetermined position in contact with the lumen wall. The balloon can then be deflated, and the catheter withdrawn.

During delivery, an endoprosthesis is typically secured to a balloon, preventing the endoprosthesis from slipping off or shifting on the catheter, which can cause loss of the endoprosthesis, and/or lead to inaccurate and imprecise delivery of the prosthesis Securement of the endoprosthesis can include mechanically clamping or crimping the endoprosthesis on the balloon.

### SUMMARY

The invention relates to systems and methods for making medical devices.

In one aspect, the invention features a method, including changing a dimension of a medical component or a medical device; and collecting a parameter associated with the medical component or the medical device as a function of time.

Embodiments may include one or more of the following features.

The medical device includes an endoprosthesis, such as a stent. The endoprosthesis includes a drug. Changing the dimension includes radially reducing the endoprosthesis. The parameter is selected from the group consisting of a force applied to the endoprosthesis, a dimension of the endoprosthesis, and a change in a dimension of the endoprosthesis. The method further includes comparing the collected parameter to a selected parameter. The method further includes validating the medical device based on a selected criterion.

The medical component includes a marker. Changing the dimension comprises radially reducing the marker. The parameter is selected from the group consisting of a force applied to the marker, a dimension of the marker, and a change in a dimension of the marker. The method further includes comparing the collected parameter to a selected parameter. The method further includes validating the medical component based on a selected criterion.

The medical device includes a medical balloon. Changing the dimension includes folding the balloon from a first configuration to a second configuration. The parameter is selected from the group consisting of a force applied to the balloon, a dimension of the balloon, and a change in a dimension of the balloon. The method further includes comparing the collected parameter to a selected parameter. The method further includes validating the medical device based on a selected criterion.

In another aspect, the invention features a system, including a first apparatus capable of changing a dimension of a medical component or a medical device; and a second apparatus operably interfaced with the first apparatus, the second apparatus being capable of collecting a parameter associated with the medical component or the medical device as a function of time.

Embodiments may include one or more of the following features. The first apparatus includes a stent crimper. The medical device includes an endoprosthesis. The endoprosthesis includes a drug. The parameter is selected from the group consisting of a force applied to the endoprosthesis, a dimension of the endoprosthesis, and a change in a dimension of the endoprosthesis. The medical component includes a radiopaque marker. The parameter is selected from the group consisting of a force applied to the marker, a dimension of the marker, and a change in a dimension of the marker. The first apparatus includes a balloon folding apparatus. The medical device includes a medical balloon. The parameter is selected from the group consisting of a force applied to the balloon, a dimension of the balloon, and a change in a dimension of the balloon.

Other aspects and features will be apparent from the description of the preferred embodiments thereof and from the claims.

### DESCRIPTION OF DRAWINGS

FIG. 1 is a diagrammatic view of an embodiment of a balloon catheter carrying a stent.

FIG. 2 is a schematic diagram of an embodiment of a system for making a medical device.

FIG. 3 is a plot of force vs. displacement.

FIG. 4 is a diagrammatic view of an embodiment of a system for making a medical device.

FIG. 5 is a schematic diagram of an embodiment of a system for making a medical device.

FIG. 6 is a schematic diagram of an embodiment of a system for making a medical device.

### DETAILED DESCRIPTION

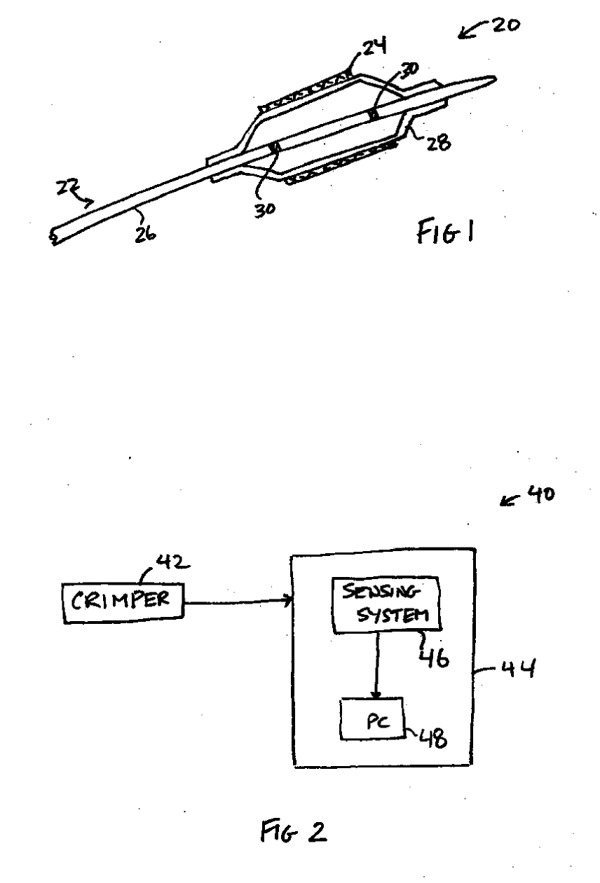

Referring to FIG. 1, a stent delivery system 20 includes a balloon catheter 22 carrying an endoprosthesis, as shown, a stent 24. Balloon catheter 22 includes a catheter shaft 26 configured to be inserted into a body, a medical balloon 28 carried by the catheter shaft, and marker bands 30 swaged to the catheter shaft. Bands 30 allow the position of balloon 28 and stent 24 to be tracked using X-ray fluoroscopy and/or magnetic resonance imaging. Prior to use, balloon 28 is folded into a compacted configuration so that it can be inserted into a vessel of the body, and stent 24 is radially crimped for a first configuration to a second, smaller configuration to the folded balloon. During use, balloon 28 can be unfolded at a target site by delivering a fluid into the interior of the balloon, thereby expanding the balloon and expanding stent 24 to contact against the wall of the vessel.

FIG. 2 shows a schematic diagram of an embodiment of a system 40 for crimping stent 24 to balloon 28. System 40 includes a stent crimper or crimping device 42 and a process parameter collection apparatus 44 operably interfaced with the stent crimper. As shown, parameter collection apparatus 44 includes a sensing system 46 that is capable of sensing one or more process parameters associated with stent 24 and/or the stent crimping process, and a computer 48 operably interfaced with the sensing system. Examples of stent crimper 42 are described in Austin, U.S. Pat. No. 6,360,577; Motsenbocker, U.S. Pat. No. 6,968,607; Williams et al., U.S. Pat. No. 5,546,646; Timmermans et al., U.S. Pat. No. 5,183,085; Cottone, Jr., U.S. Pat. No. 5,626,604; Morales, U.S. Pat. Nos. 5,725,519, 5,810,873; US-2004-0181236-A1; WO 97/20593; and WO 98/19633, all hereby incorporated by reference. Stent crimping devices are also commercially available, e.g., from Machine Solutions Inc. (Flagstaff, Ariz.). Sensing system 46 may include one or more sensors (such as transducers, force gauges, calipers, proximity sensors, fiber optic sensors, accelerometers, displacement transducers, and thermometers) capable of sensing or measuring one or more parameters associated with the crimping process as a function of time. For example, the process parameters may include the amount of force applied by crimper 42 to stent 24, the dimensional change or the amount of displacement of the stent as it is being crimped, the temperature of the stent, the size of the opening of the crimper, the change in size of the opening of the crimper, and the temperature of the crimper. Computer 48 is capable of collecting and recording as a function of time the process parameters sensed or measured by sensing system 46, and comparing the recorded parameters to selected parameters for validation purposes, as described below. An example of parameter collection apparatus 44 is commercially available from Sciemetric Instruments Inc. (Ottawa, ON, Canada) as part of the Process Signature Verification® method.

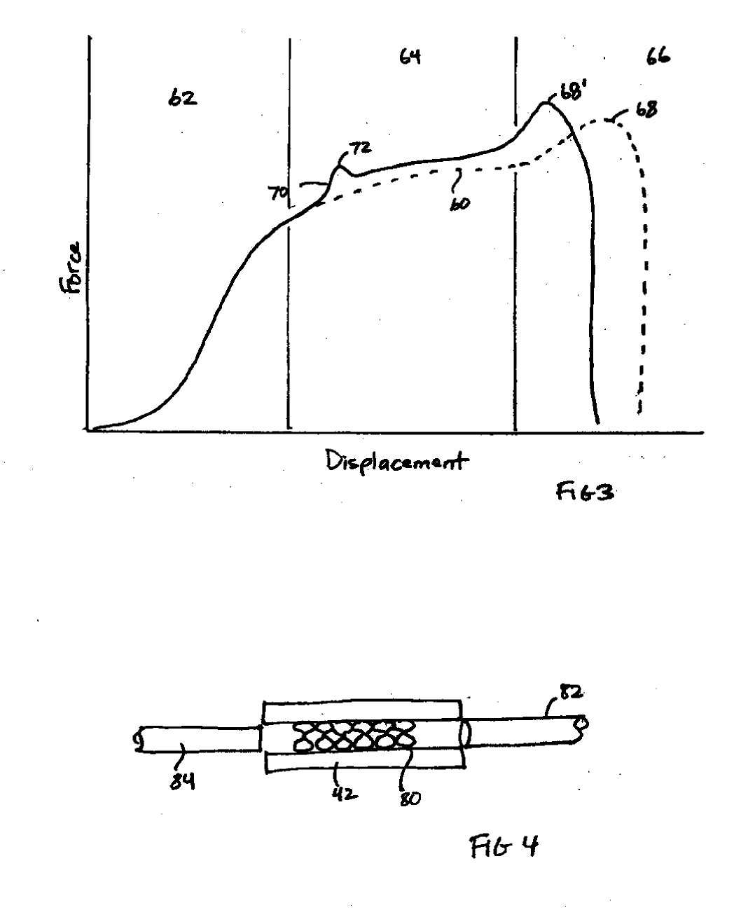

Still referring to FIG. 2, system 40 is capable of providing an in-process monitoring method that provides an objective evaluation of the success of the stent crimping process during manufacturing. For example, prior to manufacturing, system 40 can be used to provide a “normal” plot of selected process parameters for a successful crimping process that is later used for comparison during manufacturing. This normal plot can be determined empirically by crimping a meaningful number of stents, evaluating (e.g., visually and/or analytically) whether the stent crimping processes were successful, and seeing what plots the successful crimping processes generated. For example, system 40 can collect the force applied to the stent as a function of time, and the displacement (e.g., radial displacement) of the stent as a function of time to generate empirically a “normal” plot of force vs. displacement. As shown in FIG. 3, the normal plot 60 may include a force ramp region 62, a crimping region 64, and an end region 66. In force ramp region 62, there is initially substantial displacement of the stent as the crimping force is applied, until a threshold force (Fₜ) is reached, and the stent is decreasingly displaced as the crimping force is increased, for example, due to work hardening. In crimping region 64, as the crimping force is increased, the rate of displacement increases relative to the rate of displacement in force ramp region 62 because the stent is being crimped, for example, to a balloon. In end region 66, a peak 68 in the normal plot indicates the crimping force applied to secure the stent. In some embodiments, peak 68 may not be present.

During manufacturing, system 40 collects one or more sensed or measured process parameters, and records the parameters as a function of time. System 40 can then compare the recorded parameters to one or more selected sets of parameters (for example, parameters from a successful stent crimping process) to provide an indication of whether or not the stent crimping process was successful. For example, referring again to FIG. 3, during manufacturing, system 40 can again collect the force applied to the stent as a function of time, and the displacement of the stent as a function of time. From these collected process parameters, a “measured” plot 70 of force vs. displacement can be generated and compared to normal plot 60 of force vs. displacement from a comparable (e.g., identical) successful crimping process. If measured plot 70 is within a predetermined criterion (e.g., tolerance of normal plot 60), then the stent crimping process is considered to be successful, and if the measured plot is outside the predetermined tolerance of the normal plot, then the stent crimping process is considered to be unsuccessful. For example, as shown in FIG. 3, measured plot 70 includes a force peak 72 in crimping region 64 that may indicate that the stent is pinching against the balloon, and/or that struts of the stent are overlapping, both of which may require additional crimping forces to displace the stent. Peak 68′ of measured plot 70 may also indicate that the stent is displaced less than normal due to pinching and/or overlapping struts. Thus, system 40 provides a method of validating the stent crimping process during development and/or on-going process monitoring during manufacturing.

After stent 24 is crimped to balloon catheter 22, stent delivery system 20 can be used according to conventional methods. Other catheter systems are described in, for example, Wang U.S. Pat. No. 5,195,969, and Hamlin U.S. Pat. No. 5,270,086. Exemplary stent delivery techniques are known to one skilled in the art.

In other embodiments, the systems and methods described herein can be applied to other endoprostheses. For example, stent 24 can be a conventional (e.g., bare metal) stent (for example, as described in U.S. Pat. Nos. 5,725,570, 5,366,504, and 5,234,457), or the stent can also be a part of a stent-graft or a covered stent. The stent-graft or covered stent can be a stent attached to a polymer matrix, for example, a biocompatible, non-porous or semi-porous polymer matrix made of polytetrafluoroethylene PTFE), expanded PTFE, polyethylene, DACRON™, urethane, or polypropylene. The polymer matrix can include a releasable therapeutic agent or a pharmaceutically active compound, such as described in U.S. Pat. No. 5,674,242, and commonly-assigned U.S. Ser. No. 09/895,415, filed Jul. 2, 2001. The therapeutic agents or pharmaceutically active compounds can include, for example, anti-thrombogenic agents, antioxidants, anti-inflammatory agents, anesthetic agents, anti-coagulants, and antibiotics. The systems and methods described herein can be particularly useful for endoprostheses having releasable therapeutic agents or pharmaceutically active compounds because the polymer matrix can be damaged during the crimping process. In some embodiments, to reduce cross contamination and/or damage to the endoprosthesis, a protective polymer sheath is placed between the endoprosthesis and the crimper.

The systems and methods described herein can also be applied to endoprostheses that are self-expandable or a combination of self-expandable and balloon-expandable. For example, referring to FIG. 4, a self-expandable endoprosthesis 80 can be compressed as described above and in US-2004-0181236-A1, and inserted into a sheath 82 using a plunger 84. Portions of the crimper that contact the endoprosthesis can be coated with a lubricious layer, e.g., one including Teflon® (polytetrafluoroethylene), to allow plunger 84 to easily move the endoprosthesis and to reduce damage to the endoprosthesis.

While a number of embodiments have been described, the invention is not so limited.

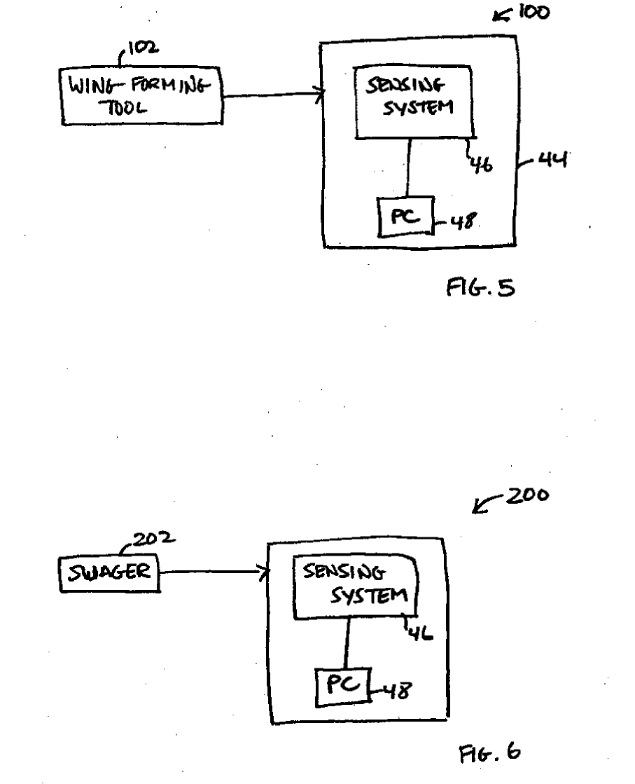

For example, the methods and systems described herein can be applied to the process of folding a medical balloon so that it can be inserted into the body. Medical balloon, such as angioplasty balloons, are typically wrapped about a catheter in a multi-lobe or winged configuration. Referring to FIG. 5, a system 100 for folding a balloon includes a wing-forming tool 102 and process parameter collection apparatus 44 operably interfaced with the wing-forming tool. An example of wing-forming tool 102 is described in U.S. Pat. No. 5,456,666, and references therein. System 100 can be used similarly to how system 40 is used to generate a “normal” plot of a successful balloon folding process, and during manufacturing, to compare corresponding “measured” plots to the normal plot to provide an in-process monitoring method that provides an objective evaluation of the success of the balloon folding process.

As another example, the methods and systems described herein can also be used to attach (e.g., swage) band(s) (e.g., marker bands 30 that are radiopaque or magnetopaque, i.e., visible by magnetic resonance imaging (MRI)) to various supports. Examples of supports include catheters, balloons, guidewires, sheath introducers, temporary filters (e.g., non-metallic, such as ceramic or polymeric, filters), stents, and grafts. In some embodiments, the band(s) can be placed on the support, e.g., slipped-fit around a polymer shaft, and the band(s) can be compressed to secure the band(s) to the support. Materials for the bands include, for example, gold, platinum, tungsten, tantalum, and metal alloys containing a sufficient percentage of heavy elements. Magnetopaque materials include, for example, non-ferrous metal-alloys containing paramagnetic elements (e.g., dysprosium or gadolinium) such as terbium-dysprosium, dysprosium, and gadolinium; non-ferrous metallic bands coated with an oxide or a carbide layer of dysprosium or gadolinium (e.g., Dy₂O₃ or Gd₂O₃); non-ferrous metals (e.g., copper, silver, platinum, or gold) coated with a layer of superparamagnetic material, such as nanocrystalline Fe₃O₄, CoFe₂O₄, MnFe₂O₄, or MgFe₂O₄; and nanocrystalline particles of the transition metal oxides (e.g., oxides of Fe, Co, Ni).

Referring to FIG. 6, a system 200 for swaging a marker band includes a swager 202 and process parameter collection apparatus 44 operably interfaced with the swager. An example of swager 202 is described in U.S. Pat. No. 6,931,899. System 200 can be used similarly to how system 40 is used to generate a “normal” plot of a successful swaging process, and during manufacturing, to compare corresponding “measured” plots to the normal plot to provide an in-process monitoring method that provides an objective evaluation of the success of the swaging process.

All publications, applications, references, and patents referred to herein are incorporated by reference in their entirety.

Other embodiments are within the claims.

What is claimed is:

1. A method, comprising:
changing a dimension of a medical component or a medical device; and
collecting a parameter associated with the medical component or the medical device as a function of time.

2. The method of claim 1, wherein the medical device comprises an endoprosthesis.

3. The method of claim 2, wherein the medical device comprises a stent.

4. The method of claim 2, wherein the endoprosthesis comprises a drug.

5. The method of claim 2, wherein changing the dimension comprises radially reducing the endoprosthesis.

6. The method of claim 2, wherein the parameter is selected from the group consisting of a force applied to the endoprosthesis, a dimension of the endoprosthesis, and a change in a dimension of the endoprosthesis.

7. The method of claim 2, further comprising comparing the collected parameter to a selected parameter.

8. The method of claim 7, further comprising validating the medical device based on a selected criterion.

9. The method of claim 1, wherein the medical component comprises a marker.

10. The method of claim 9, wherein changing the dimension comprises radially reducing the marker.

11. The method of claim 9, wherein the parameter is selected from the group consisting of a force applied to the marker, a dimension of the marker, and a change in a dimension of the marker.

12. The method of claim 9, further comprising comparing the collected parameter to a selected parameter.

13. The method of claim 12, further comprising validating the medical component based on a selected criterion.

14. The method of claim 1, wherein the medical device comprises a medical balloon.

15. The method of claim 14, wherein changing the dimension comprises folding the balloon from a first configuration to a second configuration.

16. The method of claim 14, wherein the parameter is selected from the group consisting of a force applied to the balloon, a dimension of the balloon, and a change in a dimension of the balloon.

17. The method of claim 14, further comprising comparing the collected parameter to a selected parameter.

18. The method of claim 17, further comprising validating the medical device based on a selected criterion.

19. A system, comprising:
a first apparatus capable of changing a dimension of a medical component or a medical device, and
a second apparatus operably interfaced with the first apparatus, the second apparatus being capable of collecting a parameter associated with the medical component or the medical device as a function of time.

20. The system of claim 19, wherein the first apparatus comprises a stent crimper.

21. The system of claim 19, wherein the medical device comprises an endoprosthesis.

22. The system of claim 21, wherein the endoprosthesis comprises a drug.

23. The system of claim 19, wherein the parameter is selected from the group consisting of a force applied to the endoprosthesis, a dimension of the endoprosthesis, and a change in a dimension of the endoprosthesis.

24. The system of claim 19, wherein the medical component comprises a radiopaque marker.

25. The system of claim 24, wherein the parameter is selected from the group consisting of a force applied to the marker, a dimension of the marker, and a change in a dimension of the marker.

26. The system of claim 19, wherein the first apparatus comprises a balloon folding apparatus.

27. The system of claim 19, wherein the medical device comprises a medical balloon.

28. The system of claim 27, wherein the parameter is selected from the group consisting of a force applied to the balloon, a dimension of the balloon, and a change in a dimension of the balloon.
